(19) United States
(12) Patent Application Publication
(10) Pub. No.: US 2018/0292470 A1
(43) Pub. Date: Oct. 11, 2018

(54) MINIATURE MAGNETIC FIELD DETECTOR

(71) Applicant: THE CHARLES STARK DRAPER LABORATORY, INC., Cambridge, MA (US)

(72) Inventors: James A. Bickford, Winchester, MA (US); Stephanie Lynne Golmon, Arlington, MA (US); Paul A. Ward, Dedham, MA (US); William D. Sawyer, Littleton, MA (US); Marc S. Weinberg, Needham, MA (US); John J. LeBlanc, North Andover, MA (US); Louis Kratchman, Quincy, MA (US); James S. Pringle, JR., Newton, MA (US); Daniel Freeman, Reading, MA (US); Amy Duwel, Cambridge, MA (US); Max Lindsay Turnquist, Somerville, MA (US); Ronald Steven McNabb, JR., Charlestown, MA (US); William A. Lenk, Cambridge, MA (US)

(21) Appl. No.: 15/944,234

(22) Filed: Apr. 3, 2018

Related U.S. Application Data
(60) Provisional application No. 62/482,154, filed on Apr. 5, 2017.

(51) Int. Cl. G01R 33/00; G01R 1/04; A61B 5/04; G01R 33/12

(52) U.S. Cl. CPC G01R 33/0047; G01R 1/04; G01R 33/0052; G01R 33/0023; G01R 33/1261; A61B 5/04

(57) ABSTRACT

Aspects are generally directed to a compact and low-noise magnetic field detector, methods of operation, and methods of production thereof. In one example, a magnetic field detector includes a proof mass, a magnetic dipole source coupled to the proof mass, and a substrate having a substrate offset space defined therein, the proof mass being suspended above the substrate offset space. The magnetic field detector further includes a sense electrode disposed on the substrate within the substrate offset space and positioned proximate the proof mass, the sense electrode being configured to measure a change in capacitance relative to the proof mass from movement of the proof mass in response to a received magnetic field at the magnetic dipole source. The magnetic field detector includes a control circuit coupled to the sense electrode and configured to determine a characteristic of the magnetic field based on the measured change in capacitance.

### CROSS-REFERENCE TO RELATED APPLICATION

This application claims priority under 35 U.S.C. § 119(e) to U.S. Provisional Application Ser. No. 62/482,154, titled “MINIATURE MAGNETIC FIELD DETECTOR,” filed on Apr. 5, 2017, which is hereby incorporated herein by reference in its entirety.

### BACKGROUND

Equipment that is electrically operated, or that incorporates moving structures containing electrically conductive materials or charged dielectrics, will generate static and time-varying electromagnetic fields during operation. These fields may be faint even in close proximity to the source, and will attenuate as the distance from the source is increased. Nevertheless, detectable components of these signals may exist at great distances from the source. Often great care is taken to design equipment, such as military equipment, to minimize the likelihood that unintended electromagnetic emissions will reveal the location of the equipment. Despite the care taken to reduce such emissions, low level electromagnetic signals may still exist at great distances and can be measured. Weak electromagnetic signals may also be utilized in numerous other applications, such as in communication systems, natural resource exploration, scientific research, meteorological monitoring, localization, and navigation.

### SUMMARY

Aspects and examples discussed herein are generally directed to a compact and low-noise magnetic field detector, methods of operation, and methods of production thereof. In particular, magnetic field detector designs disclosed herein may be incorporated within equipment for detecting small electromagnetic signals which emanate from equipment, vehicles, transmitters, or biophysical sources. In one example, the magnetic field detector is a microelectromechanical-based (MEMS-based) sensor which measures torsional motion of a suspended proof mass to determine one or more characteristics of a received magnetic field. Specifically, the magnetic field detector may include one or more capacitive sense electrodes which measure a variation in a charge between the proof mass and the sense electrode(s) as a result of the torsional motion of the proof mass in response to receiving the magnetic field. As further described below, particular examples may also include one or more flux concentrators, counterbalances, mechanical stop(s), and/or guard ring(s), which further improve the stability, robustness and noise performance of the magnetic field detector. Accordingly, aspects and examples discussed herein may achieve low-noise (e.g., less than 1 pT/rtHz at 10 Hz) performance at a compact size (e.g., less than 1 cm³).

According to an aspect, provided is a magnetic field detector. In one example, the magnetic field detector comprises a proof mass, a magnetic dipole source coupled to the proof mass (or a part of the proof mass), a first sense electrode positioned proximate the proof mass and configured to measure a change in capacitance relative to the proof mass from movement of the proof mass in response to a received magnetic field at the magnetic dipole source, and a control circuit coupled to the first sense electrode and configured to determine a characteristic of the magnetic field based on the measured change in capacitance.

According to another aspect, provided is another magnetic field detector. In one example, the magnetic field detector comprises a proof mass, a magnetic dipole source coupled to the proof mass, a substrate having a substrate offset space defined therein, wherein the proof mass is suspended above the substrate offset space, a first sense electrode disposed on the substrate within the substrate offset space and positioned proximate the proof mass, the first sense electrode being configured to measure a change in capacitance relative to the proof mass from torsional movement of the proof mass in response to a received magnetic field at the magnetic dipole source, and a control circuit coupled to the first sense electrode and configured to determine a characteristic of the magnetic field based on the measured change in capacitance.

As further discussed herein, in some examples, the magnetic field detector further comprises a second sense electrode coupled to the control circuit. The second sense electrode is may also be disposed on the substrate. In one example, the first sense electrode and the second sense electrode are configured to provide a differential capacitance measurement based on the change in capacitance from torsional movement of the proof mass. According to some examples, the magnetic field detector further comprises at least one support coupled to the proof mass and configured to suspend the proof mass above the substrate offset space.

According to at least one example, the magnetic field detector further comprises at least one drive electrode coupled to the control circuit and positioned proximate the proof mass, and the at least one drive electrode is configured to produce a feedback torque on the proof mass. In some examples, the at least one drive electrode is positioned on the substrate and within the substrate offset space. According to certain examples, the magnetic field detector further comprises a plurality of guard rings, each guard ring positioned to substantially surround a corresponding one of the first sense electrode or the at least one drive electrode.

According to various examples, the magnetic dipole source is formed from at least one of a static permanent magnet and an electromagnet. In some particular examples, the magnetic dipole source is a permanent magnet configured to generate a static magnetic dipole. In one example, the permanent magnet is a Neodymium Iron Boron rare Earth magnet. In certain examples, the magnetic dipole can be formed from a plurality of stacked magnets. In various examples, the magnetic dipole source is configured to generate a dynamic magnetic dipole, the control circuit being configured to provide an induced voltage to vary the dynamic magnetic dipole.

According to some examples, the magnetic field detector further comprises a counterbalance coupled to the proof mass, and the magnet coupled to a first surface of the proof mass and the counterbalance is coupled to a second surface of the proof mass distal the first surface. In some examples, the magnetic field detector further comprises at least one mechanical stop positioned to retain the proof mass within a predefined area of travel.

According to various examples, the magnetic field detector further comprises a structure wafer, and at least the proof mass and at least one support are defined in the structure wafer. In certain examples, the structure wafer is a Silicon-on-Insulator (SOI) wafer having a flexure layer, a handle layer, and an oxide layer interposed between the flexure layer and the handle layer, and the proof mass and the at least one support are defined in the flexure layer. In some examples, the magnetic field detector further comprises one or more counterbalances defined in the handle layer. In at least these examples, the structure wafer includes one or more plated holes through the oxide layer, and the one or more plated holes electrically couple the one or more counterbalances to the flexure layer.

According to various examples, the magnetic field detector further comprises a levitation suspension system configured to levitate the proof mass relative to the substrate. In particular examples, the levitation suspension system includes at least one levitation forcer positioned proximate the proof mass and configured to apply a force to maintain the proof mass at a null point, and the at least one levitation forcer is an electrostatic forcer or a magnetic forcer.

In various examples, the magnetic field detector further comprises a housing configured to enclose at least the proof mass, the first sense electrode, and the magnetic dipole and provide a vacuum environment. According to certain examples, the magnetic field detector further comprises an auxiliary sensor coupled to the control circuit and configured to measure an external parameter, the external parameter including at least one of noise, a vibration, and an ambient temperature, and wherein the control circuit is configured to adjust the characteristic of the magnetic field to compensate for an effect of the measured external parameter on the characteristic of the magnetic field.

According to various examples, in determining the characteristic of the magnetic field the control circuit is configured to determine at least a direction of the magnetic field. In various examples, the magnetic field detector further comprises a second sense electrode disposed on the substrate and within the substrate offset space, and the control circuit includes a low-noise differential sine-wave carrier generator coupled to the first sense electrode and the second sense electrode and configured to excite the first sense electrode and the second sense electrode to increase a frequency of an electronics signal produced by the received magnetic field.

In various examples, the control circuit further includes a preamplifier coupled to the first sense electrode and the second sense electrode, the preamplifier configured to provide a carrier signal amplitude-modulated by the magnetic field. In at least one example, the control circuit further includes a demodulator and a baseband filter coupled to the demodulator, the demodulator being configured to receive the amplitude-modulated carrier signal, and the baseband filter being configured to extract the characteristic of the magnetic field from an output of the demodulator. According to various examples, control circuit is further configured to apply a bias voltage to the magnetic field detector to create a negative spring force on the proof mass. In at least one example, the control circuit is further configured to apply a feedback voltage to the first sense electrode to rebalance a position of the proof mass.

According to another aspect, provided is a magnetic field transduction method. In one example, the method comprises generating a magnetic dipole on a proof mass (e.g., placing a magnet), measuring a change in capacitance between a sense electrode and the proof mass from movement of the proof mass in response to receiving a magnetic field at the proof mass, and determining a characteristic of the magnetic field based on the measured change in capacitance.

According to another aspect, provided is another magnetic field transduction method. In one example, the method comprises generating a magnetic dipole on a proof mass, the proof mass being suspended above a substrate offset space in a substrate relative to a first sense electrode disposed on the substrate, measuring a change in capacitance between the first sense electrode and the proof mass from torsional movement of the proof mass in response to receiving a magnetic field at the proof mass, and determining a characteristic of the magnetic field based on the measured change in capacitance.

According to various examples, the method further comprises providing a differential capacitance measurement from the first sense electrode and a second sense electrode based on the change in capacitance from the torsional movement of the proof mass. In some examples, the method further comprises suspending the proof mass relative to the sense electrode with at least one of one or more supports, one or more rotational bearings, an electrostatic suspension, or a magnetic suspension.

In various examples, the method further comprises providing a feedback torque on the proof mass with one or more drive electrodes positioned proximate the proof mass. In at least one example, generating the magnetic dipole includes forming the magnetic dipole on the proof mass with a permanent magnet or an electromagnetic.

According to various examples, the method further comprises counterbalancing the proof mass with a counterbalance coupled to the proof mass. In certain examples, the method further comprises measuring at least one of internal noise, external noise, an external vibration, and an ambient temperature, and correcting the characteristic of the magnetic field to compensate for the at least one of the internal noise, external noise, the external vibration, and the ambient temperature.

In certain examples, determining the characteristic of the magnetic field includes determining at least a direction of the magnetic field. According to various examples, the method further comprises exciting the first sense electrode and a second sense electrode with a low-noise differential sine-wave carrier generator coupled to the first sense electrode and the second sense electrode to increase a frequency of an electronics signal produced by the received magnetic field. In at least one example, exciting the first sense electrode and the second sense electrode with a low-noise differential sine-wave carrier generator includes generating and applying a carrier signal to the first sense electrode and the second sense electrode. In some examples, the method further comprises amplitude modulating the carrier signal with magnetic field information of the received magnetic field to generate an amplitude-modulated carrier signal, and demodulating the amplitude-modulated carrier signal and extracting the characteristic of the magnetic field from the demodulated carrier signal.

According to various examples, the method further comprises applying a bias voltage to create a negative spring force on the proof mass. In certain examples, the method further comprises applying a feedback voltage to the first sense electrode to rebalance a position of the proof mass. In other examples, the negative spring is formed by the magnetic dipole attached to the proof-mass and surrounding magnetic material.

According to an aspect, provided is a method for fabricating a magnetic field detector. In one example, the method comprises defining at least one substrate offset space in a substrate wafer, forming a first sense electrode on the substrate wafer and within the substrate offset space, defining a proof mass and at least one support in a structure wafer and suspending the proof mass by the at least one support to allow torsional movement of the proof mass, providing a magnetic dipole source on the proof mass, and coupling the substrate wafer and the structure wafer to position the proof mass proximate the substrate offset space of the substrate wafer and within capacitive communication with at least the first sense electrode.

According to various examples, the method further comprises providing the structure wafer, and the structure wafer includes a flexure layer, a handle layer, and an oxide layer interposed between the flexure layer and the handle layer. In at least one example, defining the proof mass and the at least one support in the structure wafer includes etching the flexure layer to form the proof mass and the at least one support. In some examples, the method further comprises selectively removing a first portion of the oxide layer exposed through the flexure layer. In at least one example, the method further comprises defining one or more counterbalances in the handle layer. In some examples, the method further comprises applying a metallic layer to one or more holes defined in the flexure layer to electrically couple the flexure layer and the handle layer of the structure wafer. In at least one example, the method further comprises selectively removing a second portion of the oxide layer exposed through the handle layer.

In various examples, the method further comprises applying one or more metallic bumps to a surface of the first sense electrode. According to various examples, the method further comprises forming a second sense electrode, a first drive electrode, and a second drive electrode on the substrate wafer and within the substrate offset space. In certain examples, forming the first sense electrode, the second sense electrode, the first drive electrode, and the second drive electrode on the baseplate wafer includes depositing a conducting material on a surface of the substrate wafer.

According to certain examples, providing the magnetic dipole on the proof mass includes providing the magnetic dipole source on the proof mass within a vacuum environment. In certain examples, the method further comprises varying a magnetic dipole formed via an active excitation signal within a conductive loop structure.

According to another aspect, provided is another magnetic field detector. In one example, the magnetic field detector comprises a proof mass, a magnetic dipole source coupled to the proof mass, a first sense electrode configured to measure a change in capacitance relative to the proof mass from torsional movement of the proof mass in response to a received magnetic field, a levitation suspension system configured to levitate the proof mass relative to the first sense electrode, and a control circuit coupled to the first sense electrode and configured to determine a characteristic of the magnetic field based on the measured change in capacitance.

In various examples, the levitation suspension system includes at least one levitation forcer positioned proximate the proof mass and configured to apply a force to maintain the proof mass at a null point. According to certain examples, the at least one levitation forcer is one of an electrostatic forcer and a magnetic forcer.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Any embodiment disclosed herein may be combined with any other embodiment in any manner consistent with at least one of the objectives, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment. Various aspects, embodiments, and implementations discussed herein may include means for performing any of the recited features or functions.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the disclosure. In the figures, each identical or nearly identical component that is illustrated in various figures is represented by a like numeral. For purposes of clarity, not every component may be labeled in every figure. In the figures:

FIG. 1 is a perspective view of a magnetic field detector, shown with a housing detached from the detector, according to examples discussed herein;

FIG. 2 is perspective view of the magnetic field detector illustrated in FIG. 1 with the housing attached, according to examples discussed herein;

FIG. 3 is another perspective view of components of the magnetic field detector illustrated in FIG. 1, according to examples discussed herein;

FIG. 4 is a graph demonstrating the improved noise sensitivity of a magnetic field detector system according to aspects described herein;

FIG. 5 is a plan view of the sense electrodes and drive electrodes of the magnetic field detector illustrated in FIG. 1, according to examples discussed herein;

FIG. 6 is a block diagram of a control circuit according to examples discussed herein;

FIG. 7A-7C is a process flow for fabricating a magnetic field detector, according to examples discussed herein;

FIGS. 8A-8C show a state of a magnetic field detector during each act of the process flow of FIG. 7A-7C, according to examples discussed herein;

FIG. 9 is an axial view of a proof mass and levitation forcers, according to various examples discussed herein; and

FIG. 10 is a side profile view of a levitation suspension system including the levitation forcers of FIG. 9, according to various examples discussed herein.

### DETAILED DESCRIPTION

Aspects and embodiments are generally directed to magnetic field detector systems and methods for exploiting the magnetic field component of electromagnetic signals. Systems may include one or more magnetic field detectors capable of detecting a magnetic field generated by equipment or natural processes that generate electromagnetic fields. Systems may also include one or more magnetic field detectors capable of detecting bio-physical signals generated by the body of a patient or user, such as the magnetic fields of his or her brain, heart, nerves or muscles.

Current magnetic field detectors include high noise sensors that inhibit the observation of weak magnetic field signals at low frequencies, or low noise sensors which are difficult to practically implement. For example, superconducting quantum interference devices (SQUID) require operation at cryogenic temperatures. While various atomic sensors can provide low noise performance, they are challenging to operate with low noise performance as a result of the Earth's large background magnetic field. Similarly, inductive search coils experience high noise at low frequencies. Moreover, each of these solutions is large in size and physically restrictive, which is not practical in most military or mobile applications. Accordingly, certain aspects and embodiments provide improved magnetic field detection systems and methods, as discussed below.

In one example, the magnetic field detector is a microelectromechanical-based (MEMS-based) magnetic field detector which measures a torque on a suspended proof mass to determine one or more characteristics of a received magnetic field. In particular, a magnetic dipole is generated on the proof mass by placing a magnetic dipole source (e.g., permanent magnet), such as a Neodymium Iron Boron rare Earth magnet, on the proof mass. The induced magnetic dipole generates a torque on the proof mass when exposed to an external magnetic field. The torque induces torsional motion in the proof mass, which causes a capacitance between one or more sense electrodes and the proof mass to change. The change in capacitance may then be measured to estimate one or more characteristics of the external magnetic field. In one example, the measured characteristic is a direction, in other examples, the measured characteristics is a magnitude or a phase.

It is to be appreciated that examples and/or embodiments of the apparatus and methods discussed herein are not limited in application to the details of construction and the arrangement of components set forth in the following description or illustrated in the accompanying drawings. The apparatus and methods are capable of implementation in other embodiments and of being practiced or of being carried out in various ways. Examples of specific implementations are provided herein for illustrative purposes only and are not intended to be limiting. In particular, acts, elements and features discussed in connection with any one or more examples and embodiments are not intended to be excluded from a similar role in any other example or embodiment. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, above and below, and vertical and horizontal are intended for convenience of description, not to limit the present systems and methods or their components to any one positional or spatial orientation.

The accompanying drawings are included to provide illustration and a further understanding of the various aspects and examples, and are incorporated in and constitute a part of this disclosure. The drawings, together with the remainder of the disclosure, serve to explain principles and operations of the described and claimed aspects and examples.

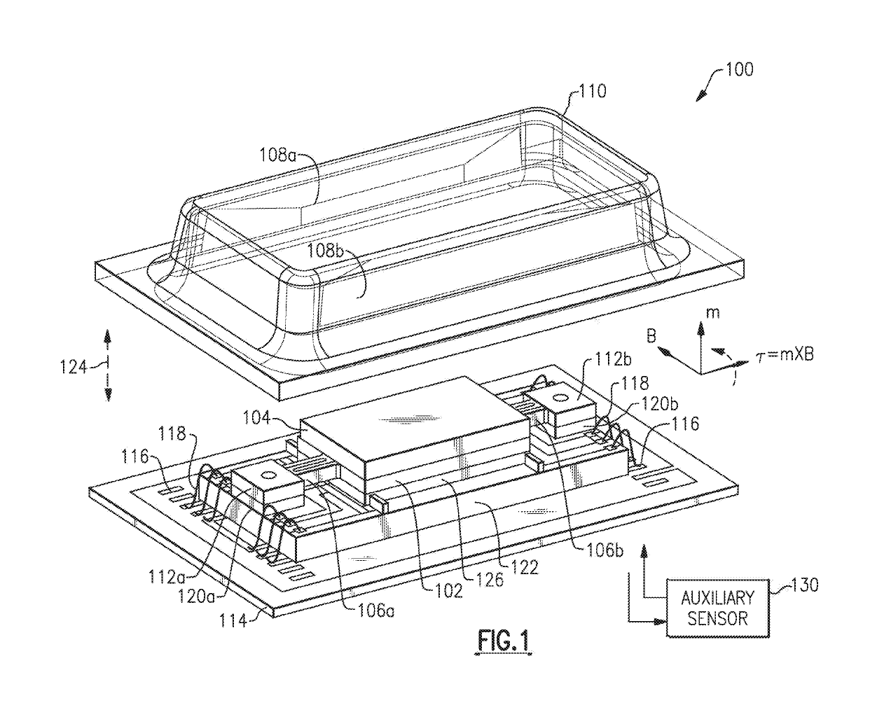

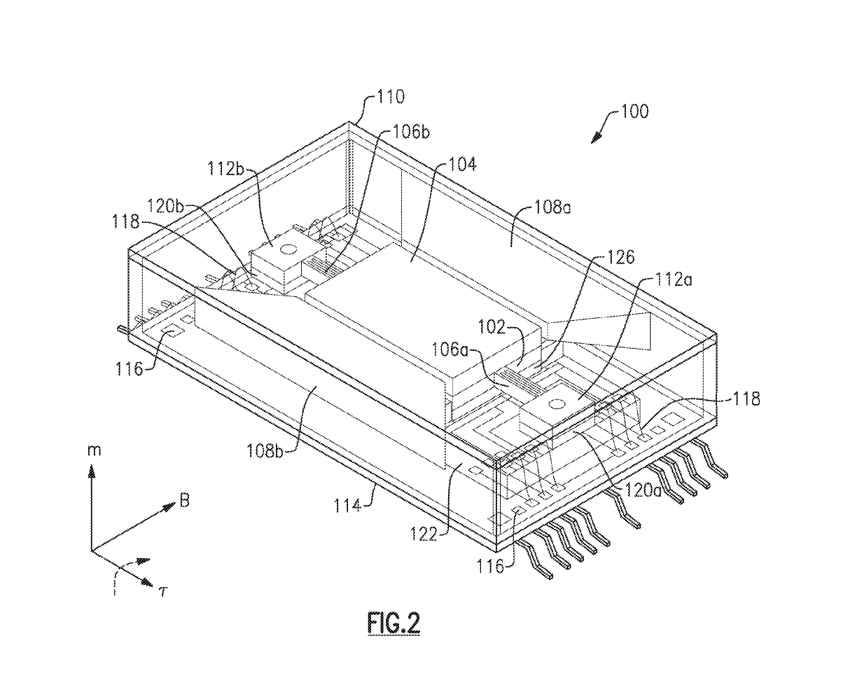

FIGS. 1 and 2 each illustrate a perspective view of a magnetic field detector 100 according to various examples described herein. FIG. 1 illustrates a view of the detector 100 with a housing 110 detached from the detector 100, and FIG. 2 shows a view of the detector 100 with the housing 110 attached. The housing 110 may be removed in a vertical direction away from the detector 100 (e.g., direction 124), as shown in FIG. 1. In FIGS. 1 and 2, the magnetic field detector 100 includes a microelectromechanical-based (MEMS-based) resonator, which may be defined by processing a structure wafer (e.g., a Silicon-on-Insulator wafer) to a desired geometry. As shown, the detector 100 may include a proof mass 102 coupled to a magnetic dipole source 104, a plurality of supports 106a, 106b (collectively “supports 106”) one or more flux concentrators 108a, 108b (collectively “flux concentrators 108”), a housing 110, one or more anchors 112a, 112b (collectively “anchors 112”), a baseplate 114, one or more electrical contacts 116, one or more leads 118, and a substrate 122, among other components. While not shown in FIGS. 1 and 2, each of the contacts 116 may couple the magnetic field detector 100 to a control circuit, examples of which are further discussed herein. In certain examples, the structure wafer is processed (e.g., etched) to define the proof mass 102, the plurality of supports 106, and the one or more anchors 112. In further examples, the magnetic field detector 100 may also include one or more counterbalances 126 that are coupled to the proof mass 102. In certain examples, the magnetic field detector 100 may also include one or more sense electrodes and one or more drive electrodes, each of which are positioned on the substrate 122 and obscured in FIGS. 1 and 2 by the counterbalance 126. As shown, the substrate is positioned on the baseplate 114.

In various examples, the magnetic field detector 100 determines one or more characteristics of a received magnetic field, which in one example is a bio-electrical signal, based on measured capacitance variations due to torsional motion of the proof mass 102 in response to receiving the magnetic field. While in some examples, a combination of linear forces may result in the torsional motion of the proof mass 102, in certain other examples, a variation in capacitance as a result of a single linear force may be measured. The proof mass 102 is supported by the plurality of supports 106, each of which form a rotationally compliant spring anchored to the substrate 122 via a respective anchor 112a, 112b. In the shown example, each support 106 is a flexured beam interposed between a side surface of the proof mass 102 and a corresponding anchor 112a, 112b. That is, a first support 106a is interposed between a first side surface of the proof mass 102 and a first anchor 112a, and a second support 106b is interposed between a second side surface of the proof mass 102 and a second anchor 112b. Each anchor 112 is coupled to the substrate 122 with a respective anchor ground 120a, 120b. The first anchor 112a is coupled to the substrate 122 at the first anchor ground 120a, and the second anchor 112b is coupled to the substrate 122 at the second anchor ground 120b.

As shown in FIG. 1, the first support 106a and the second support 106b may be coupled to opposing sides of the proof mass 102. The dimensions of the supports 106 are selected such that the overall stiffness of the supports 106 are sufficient to withstand operational shock loads while maximizing a response to input torques. While shown as including a pair of supports 106a, 106b, in various other examples the magnetic field detector may include one (e.g., in a “lever” arrangement) or any number of supports 106. For instance, the detector 100 may include three supports 106, or an arrangement of four or more supports 106.

In various other examples, the proof mass 102 may be levitated by an electrostatic suspension, levitated by an electromagnetic suspension, and/or suspended by an equivalent rotational bearing. Unlike the example illustrated in FIG. 1, in these examples it may be advantageous to design the proof mass 102 (and/or the magnetic dipole source 104) to have a circular or cylindrical shape to permit rotation thereof. In such an example, the levitated proof mass (e.g., relative to a substrate) is positioned to move (e.g., rotate) with very low resistance and low stiffness. Such an arrangement may maximize a scale factor of the magnetic field detector 100 while retaining a structural stability and robustness. In such an example, the electrostatic suspension, electromagnetic suspension, and/or rotational bearing may supplement the one or more illustrated flexured beams of FIG. 1 (e.g., supports 106) or replace the one or more flexured beams.

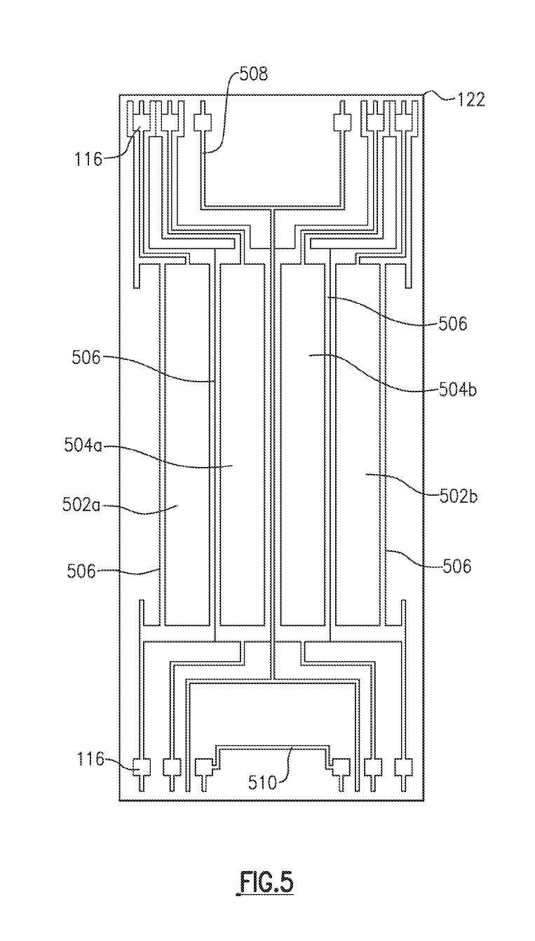

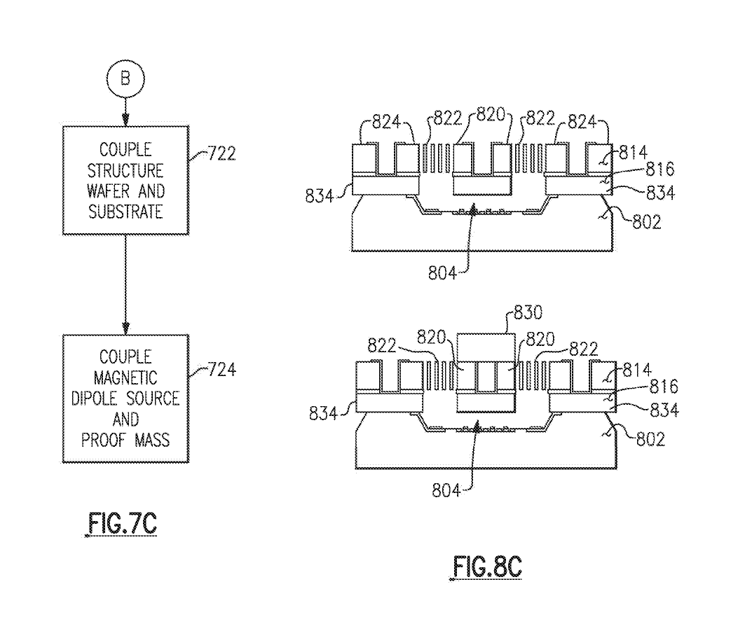

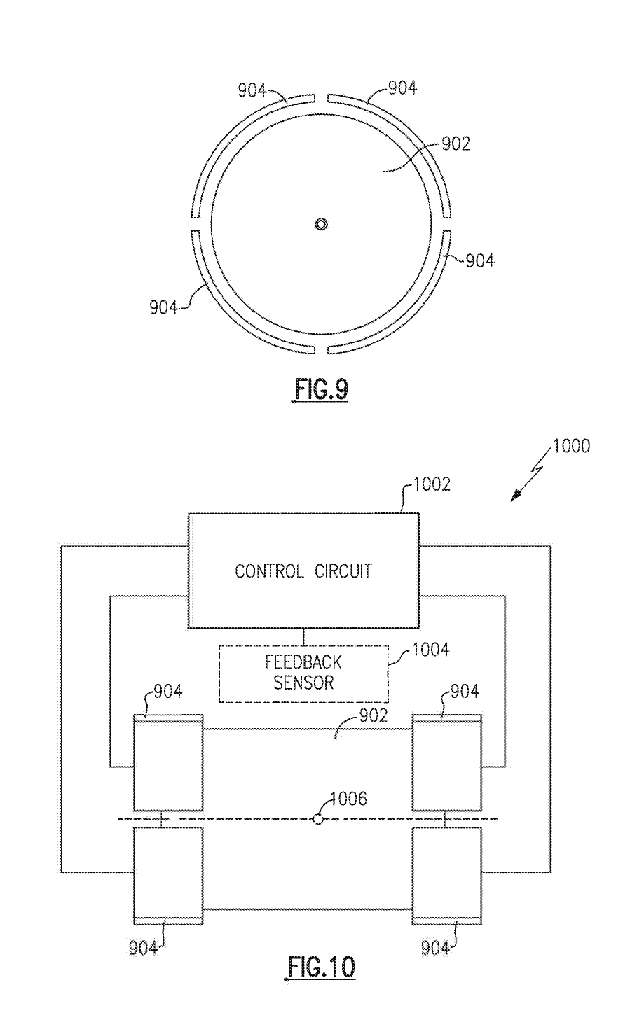

One example of a levitation suspension system 1000 is described with reference to FIG. 9 and FIG. 10. In particular, FIG. 9 illustrates an axial view of a proof mass 902 and levitation forcers 904, and FIG. 10 illustrates a profile view of a levitation suspension system 1000 that includes the levitation forcers 904 of FIG. 9. Examples of the levitation suspension system 1000 may be incorporated within any of the examples of the magnetic field detectors described herein, such as the magnetic field detector 100 described with reference to FIG. 1 and FIG. 2. That is, the proof mass 902 may be the proof mass 102 illustrated in FIG. 1. As shown, the levitation suspension system 1000 may include one or more levitation forcers 904 that apply a levitating force to the proof mass 902 to levitate the proof mass against gravity and other induced forces. In certain examples, each of the one or more levitation forcers 904 may include one or more of the sense electrodes 502 or drive electrodes 504 further described below with reference to FIG. 5. While in certain examples, each levitation forcer 904 may be an electrostatic forcer (e.g., for electrostatic levitation), in various other examples, each levitation forcer 904 may be a magnetic forcer (e.g., for magnetic levitation).

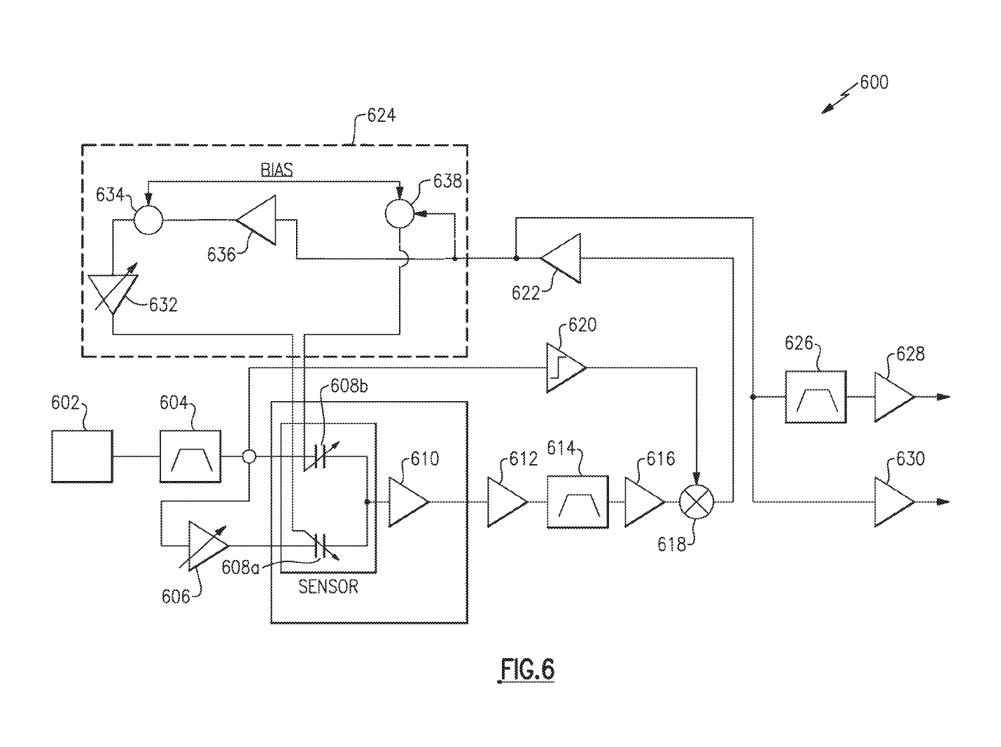

A control circuit 1002 (e.g., control circuit 600 illustrated in FIG. 6) coupled to the levitation forcers 904 receives feedback from each levitation forcer 904 and/or one or more feedback sensors 1004. If a position of the proof mass 902 is displaced relative to a desired null point (e.g., shown as point 1006), the control circuit 1002 provides a control signal to one or more of the levitation forcers 904 to increase or decrease the force applied by the receiving levitation forcer 904 and return the proof mass 902 to the null position. In certain examples, the proof mass 902 may be metalized (e.g., at an end of the proof mass) to increase the sensitivity of the proof mass 902 to the levitation force. The position of the proof mass 902 (relative to the null position) may be capacitively measured based on a capacitance between the proof mass 902 and one or more sense electrodes (e.g., sense electrodes 502 described with reference to FIG. 5).

The number and arrangement of levitation forcers 902 may be selected based on the desired application of the corresponding magnetic field detector. While FIG. 9 illustrates a plurality of levitation forcers 904 (e.g., four) radially aligned about the circumference of an axial proof mass 902, various other arrangements are possible. In particular, the number, shape, and arrangement of levitation forcers 904 may depend on the particular shape of the proof mass 902 and packaging constraints (e.g., size, weight, available space, etc.). In addition to maintaining the proof mass 902 at a desired null position, in certain instances, the levitation forcers 904 may be used to rotate the proof mass 902 at a desired velocity, or reposition the proof mass 902 to a desired orientation. In addition to assessing the position of the proof mass 902 relative to a null position, one or more signals from the illustrated feedback sensor 1004 may be used by the control circuit 1002 to infer external stimuli that induce proof mass 902 movement. The feedback sensor 1004 may be an optical sensor, an accelerometer, a capacitive sensor, or any other type of position sensor.

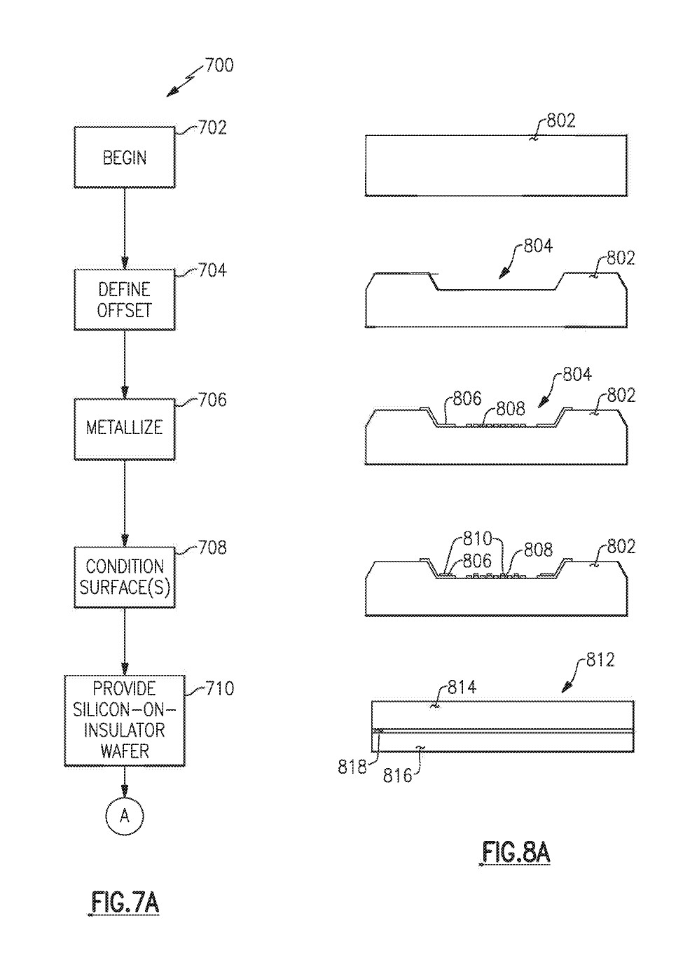

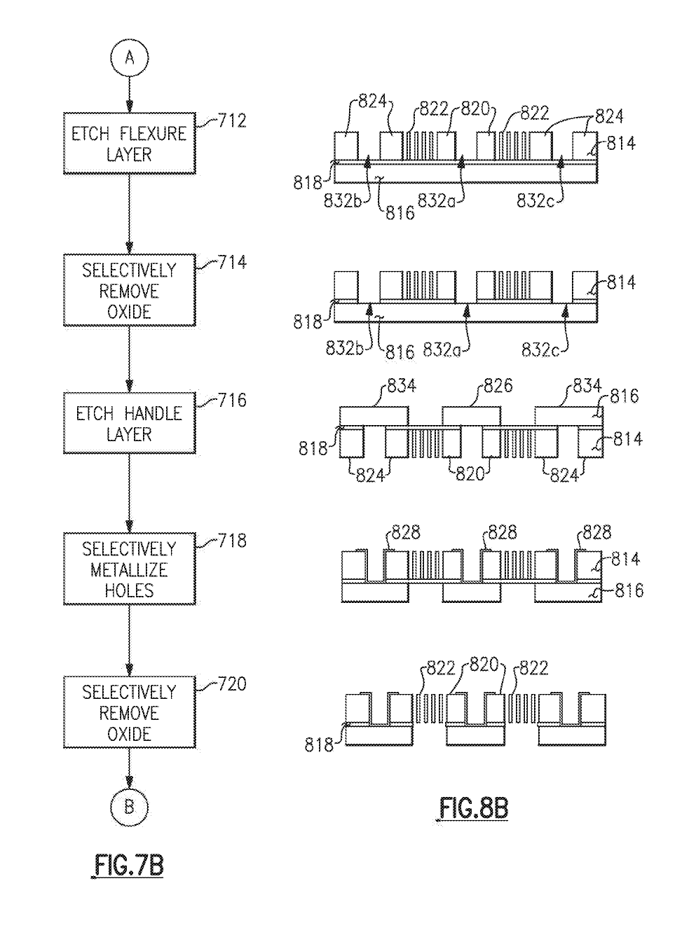

Referring to FIGS. 1 and 2, in various examples, the plurality of supports 106 may suspend the proof mass 102 above a substrate offset space defined in the substrate 122. That is, the substrate 122 may include an area (referred to as a “substrate offset space”) formed in a surface thereof beneath the proof mass 102 (e.g., and counterbalance 126 shown in FIGS. 1 and 2). The substrate offset space is obscured in FIGS. 1 and 2 by the counterbalance 126. While described as being suspended “above” the substrate offset space, in other examples, the proof mass 122 may be partially positioned within the substrate offset space. In other examples, the proof mass 102 may be positioned in close proximity to the substrate offset space but not directly above or within the substrate offset space. As discussed, in certain examples, the magnetic field detector 100 may include one or more sense electrodes and one or more drive electrodes, each of which are positioned on the substrate 122 and in capacitive communication with the proof mass 102. In particular, each of the sense electrodes and the drive electrodes may be positioned within the substrate offset space and may form a sense gap with the proof mass 102. In certain examples, the substrate offset space is formed by etching the substrate 122; however, other processing techniques may be used to form the substrate offset space, such as milling, grinding, or one or more deposition processes. Various aspects of a substrate, a substrate offset space, sense electrodes, and drive electrodes are discussed below with reference to at least FIG. 7A-7C and FIGS. 8A-8C.

In various examples, an impinging magnetic field concentrated on the magnetic dipole source 104 generates a torque and effects motion of the proof mass 102. For instance, the torque, τ, may be represented as:

τ=M×B

where, M, is the strength of the magnetic dipole provided by the magnetic dipole source 104 (e.g., A-m²) and, B, is the strength of the received magnetic field (e.g., in Tesla).

In many instances, the proof mass 102 responds to the torque by rotating about a torque axis (shown as axis τ in FIGS. 1 and 2). In one example, the rotation can be represented as:

$$θ = \frac{τ}{( {Is}^{2} ) + ( Ds ) + k}$$

where, θ, is the angle of rotation, τ, is the torque, I, is the polar moment of inertia, s, is the complex frequency, D, is a damping coefficient, and k is the rotational stiffness. In this way, the torque generated from the magnetic field induces motion in the proof mass 102, which reacts against the stiffness of the supports 106 (or the levitation suspension system 1000).

In various examples, the rotation of the proof mass 102 increases or decreases the distance between the proof mass 102 and the sense electrode(s) positioned on the substrate 122. As the distance between the proof mass 102 and the sense electrode(s) increases or decreases, the relative capacitance between the sense electrode(s) and the proof mass 102 varies. The resulting change in capacitance can be measured by the electronics to estimate the characteristic(s) of the received magnetic field. For example, this may include a direction (or directions), phase, and/or a magnitude. In various examples, the magnetic field detector 100 may include a plurality of electrical leads 118, at least one of which couples a sense electrode to a corresponding contact 116. Each electrical contact 116 may connect the corresponding lead 118 to the control circuit, which may determine the direction, the magnitude, and/or the phase of the received magnetic field based on the sensed variation in capacitance. As illustrated, the substrate 122 may be coupled to the baseplate 114. Accordingly, the baseplate 114 supports the substrate 122, as well as other components of the detector 100, and may include one or more fasteners for creating a seal with the housing 110.

In certain examples, the control circuit may also send one or more control signals to the electrical contacts 116 and the corresponding leads 118. In particular, the control circuit may generate one or more control signals which can be used charge one or more drive electrodes and produce a feedback torque on the proof mass 102. That is, the magnetic field detector 100 may further include one or more drive electrodes positioned on the substrate 122 (e.g., within the substrate offset space) which rebalance the proof mass 102 to a nominal rotational position based on a received control signal. Such an arrangement may reduce non-linearities in the capacitance measurements (e.g., from the supports 106) while also extending the dynamic range of the magnetic field detector 100. In such an example, a lead 118 may receive the control signal from a contact 116 and provide the control signal to a drive electrode.

In certain examples, the magnetic field detector 100 may include a magnetic dipole source 104 (e.g., a permanent magnet) which produces an electric dipole at the proof mass 102. In the example shown in FIG. 1, the magnetic dipole source 104 is coupled to a top surface of the proof mass 102; however, in certain other examples, the proof mass 102 itself may be composed of a magnetic material. That is, while shown as separate components in at least FIGS. 1 and 2, in certain other examples, the magnetic dipole source 104 and the proof mass 102 may be the same component.

In various examples, the magnetic dipole source 104 includes one or more magnetic material(s). For example, the magnetic material(s) may include one or more permanent magnetic materials such as rare Earth magnets, ferrite magnets (e.g., Neodymium Iron Boron, Samarium Cobalt, or Alnico), or other hard magnetic materials. Alternatively, the magnetic dipole source may be formed from a time-varying magnetic material, such as one or more soft magnetic material(s) (e.g., Magnesium Zinc Ferrite) excited by an external source via an excitation signal of a predefined frequency. Further examples of the magnetic dipole source may include a series of two or more stacked magnets or a plurality of magnets arranged in a predetermined order. To increase the strength of the magnetic dipole, and therefore increase the sensitivity of the detector 100 to magnetic fields, micron-thick layers of magnets may be stacked together.

In some particular examples, the magnetic dipole source 104 may generate a variable magnetic dipole. For instance, the magnetic dipole source 104 may include and drive an electromagnet to generate a time-varying magnetic dipole. In such an example, the control circuit may continuously, or periodically, drive a current to the magnetic dipole source 104 to produce a dynamic magnetic dipole. However, in other examples, the control circuit may continuously, or periodically, drive a current to the magnetic dipole source 104 to produce a static magnetic dipole. In the various examples in which the magnetic dipole source is configured to generate a dynamic magnetic dipole, the control circuit may be configured to provide an induced voltage to vary the dynamic magnetic dipole. Such operation may be useful to accommodate various changes in operating conditions, among offering other benefits. Specifically, the control circuit may drive the drive electrodes at an alternating-current (AC) frequency such that the detector 100 up-converts (e.g., increases a frequency) the received magnetic field information to a frequency above a 1/f noise limit, improving the performance of the detector 100.

As illustrated in at least FIGS. 1-2, in at least one example the proof mass 100, the supports 106, and the anchors 112a, 112b are defined in a same structure wafer. For instance, the structure wafer may include a Silicon-on-Insulator wafer having a flexure layer, a handle layer, and an oxide layer. The oxide layer may be interposed between the flexure layer and the handle layer. As further described with reference to FIG. 7A-7C and FIGS. 8A-8C, the proof mass 102, the supports 106, and the anchors 112a, 112b may be defined in the flexure layer. It is appreciated that in some instances, the magnetic dipole source 104 and/or an intervening material (e.g., a glue or other adhesive material) between the magnetic dipole source 104 and the proof mass 102 may introduce an asymmetry in a balance of the proof mass 102. Such an asymmetry may generate undesired sensitivities to external accelerations. In certain examples, the magnetic field detector 100 may include the one or more counterbalances, such as the counterbalance 126, to compensate for the asymmetry.

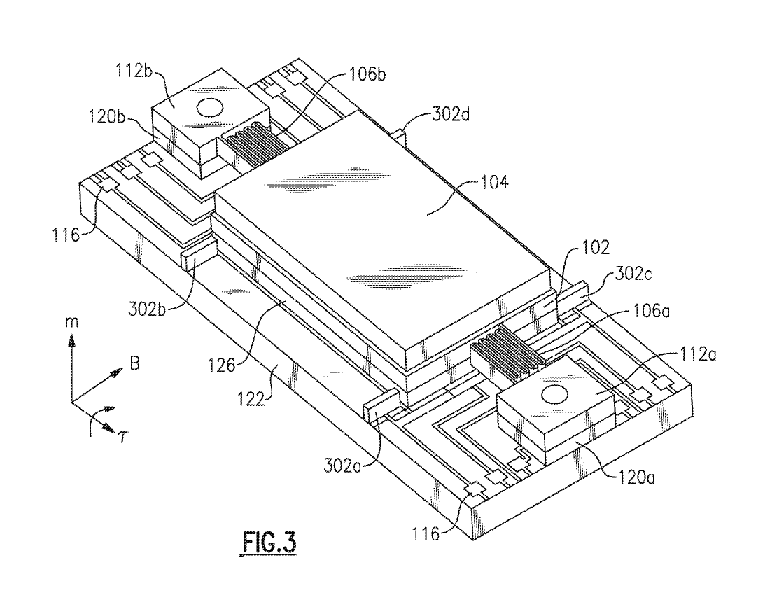

In various examples, the magnetic field detector 100 may alternatively or additionally compensate for the external accelerations, and/or effects from other external parameters, by directly measuring the external parameter with an auxiliary sensor, and adjusting the measured magnetic field to compensate for the external parameter. For instance, in addition to external accelerations, the auxiliary sensor may measure at least one of noise, ambient temperature, or vibrations. Accordingly, the auxiliary sensor may be an accelerometer, temperature sensor, or noise sensor, to name a few examples. The control circuit may receive measurements from the auxiliary sensor use various filtering techniques (e.g., digital signal processing filter techniques), for example, to adjust the characteristic of the magnetic field to compensate for the effect(s) of the measured external parameter on the measured characteristic of the magnetic field. In various examples, adjusting the measured characteristic of the magnetic field may include applying a filter to remove the effect of the external parameter. The particular arrangement and position of auxiliary sensors within the magnetic field detector 100 may vary based on the particular external parameter desired to be measured, as well as, the particular architecture of the magnetic field detector 100 itself. Accordingly, an auxiliary sensor is generally represented by auxiliary sensor block 130 in FIG. 1 (removed in FIG. 2 and FIG. 3).

Referring to FIG. 3, there is illustrated a view of the magnetic field detector 100 shown in FIGS. 1 and 2 with at least the housing 110 and the baseplate 114 removed. In FIG. 3, a counterbalance 126 is positioned on a bottom surface of the proof mass 102 and also suspended above the substrate offset space. The counterbalance 126 reduces the pendulosity of the proof mass 102 and, therefore, a sensitivity of the proof mass 102 to undesired inputs, such as vibrations. In further examples, mechanical stops 301, 302, 302c, 302d may be coupled to the counterbalance 126 to prevent large excursions of the proof mass 102 from a predefined area of travel. That is, the mechanical stops 302a, 302b, 302c, 302d may be positioned to define a limit of travel of the proof mass 102 relative to the substrate 122 and within the detector 100. For example, FIG. 3 shows each of the mechanical stops 302a, 302b, 302c, 302d coupled to a side surface of the counterbalance 126. While shown as having one of the mechanical stops 302a, 302b, 302c, 302d at each corner of the rectangular counterbalance 126, in various other examples, the mechanical stops 302a, 302b, 302c, 302d may be positioned at other locations on the counterbalance 126, or may be attached to the housing 110.

Returning to FIGS. 1 and 2, the flux concentrators 108 can operate to focus the received magnetic field on the magnetic dipole source 104. As shown, the flux concentrators 108 may be integrated within the housing 110, and in particular, attached to an interior surface of the housing 110. In other examples, the flux concentrators 108 may be attached to the substrate 122 or the baseplate 114. In various examples, the flux concentrators 108 magnify the intensity of the magnetic field near the location where the magnetic field intercepts the magnetic dipole. The flux concentrators 108 may each be composed of soft magnetic material, having a high magnetic permeability, which routes the flux through a spatial volume thereof. For example, each flux concentrator 108 may be composed a soft ferrite. By positioning the flux concentrators 108 near the magnetic dipole source 104, the magnetic field is concentrated to provide a gain at the magnetic dipole source 104. In the shown example, a first flux concentrator 108a is positioned proximate a side surface of the proof mass 102 and a second flux concentrator 108b is positioned proximate another, distal, side surface of the proof mass 102.

In various examples, each flux concentrator 108 is positioned as close as possible to the magnetic dipole source 104 to maximize the provided gain. The performance of each flux concentrator 108 may also be enhanced by increasing a length and/or an area of the respective flux concentrator 108 to maximize the amount of flux received and directed to the magnetic dipole source 104. Relative to the housing 110, each flux concentrator 108 may be internal, external, or a combination of both depending upon the level of enhancement desired. In addition to the flux concentrators 108, in certain examples the magnetic field detector 100 may include additional signal processing components which enhance the ability of the magnetic field detector 100 to resolve small signals. Such components are further described below with reference to at least FIG. 6. According to certain other examples, the one or more sense electrodes and the one or more drive electrodes that provide the capacitive readout may be replaced by other structures that are configured to measure the torque on the proof mass 102 from a received magnetic field. For instance, the magnetic field detector 100 may include one or more sensors that measure the torque by its effect on a frequency of one or more of the plurality of supports 106, or one or more sensors that optically measure a displacement of the proof mass 102.

As also shown in FIGS. 1 and 2, in various examples the magnetic field detector 100 includes the housing 110. The housing 110 is positioned to encompass the other components of the magnetic field detector 100, such as the proof mass 102, the plurality of supports 106, the one or more flux concentrators 108, the one or more anchors 112, the substrate 122, the sense electrodes, the drive electrodes, and the one or more electrical contacts 116, among other components. In certain examples, the housing 110 may provide a vacuum environment which reduces the sensitivity of the magnetic field detector 100 to acoustic coupling and air damping, which reduces Brownian noise. In addition to these benefits, the housing 110 protects the discussed components of the magnetic field detector 100 from dust, moisture, and other contaminants. In one example the housing 110 may be formed from transparent glass to permit displacement of the proof mass 102 to be measured optically.

According to an example, a scale factor of the magnetic field detector 100 may be increased by using one or more bias voltages to create an electrostatic spring with a negative stiffness relative to the mechanical stiffness of the supports 106. A strong bias voltage on a sense electrode, drive electrode, and/or other electrodes positioned near the proof mass 102 and/or source of concentrated charge 104 generates a force (e.g., negative spring force) which is opposite of the mechanical spring force of the supports 106, and thereby decreases the overall stiffness of the MEMS structure. Accordingly, when summed, the negative stiffness reduces the total stiffness of the magnetic field detector 100 and increases the response of the proof mass 102 to a received magnetic field. Such an approach provides the benefit of increased performance without the loss of robustness, which would otherwise result if the stiffness of each of support 106 was mechanically reduced. While in certain examples the magnetic field detector 100 may include additional electronics to create a negative spring by force inputs (e.g., a control loop or a magnetic field), application of bias voltages to create an electrostatic spring provides the benefit of low-noise performance and reduced complexity. The force between the magnetic dipole source 104 and additional electronics placed on the substrate or other location nearby, can introduce a negative spring stiffness due to the force between the magnetic dipole and additional electronics as the proof-mass 102 rotates.

As discussed herein, multiple magnetic field detectors 100 may be integrated into an array to enhance magnetic field detection performance. That is, an array of magnetic field detectors may be arranged to improve the ability of each individual detector to sense weak magnetic field signals and/or to measure a spatial distribution of magnetic fields around a piece of equipment. It is appreciated that other implementations may be designed to detect bio-physical signals generated by other areas of the body of a patient or user, such as the heart, nerves, or muscles, to name a few examples. Auxiliary sensors may also be incorporated within the electronics to measure effects which may introduce errors in the intended measurement (e.g., one or more external parameters). For example, inertial sensors and/or temperature sensors can be co-located with the magnetic field detectors to measure magnetic fields, accelerations (e.g., platform movement), or temperature.

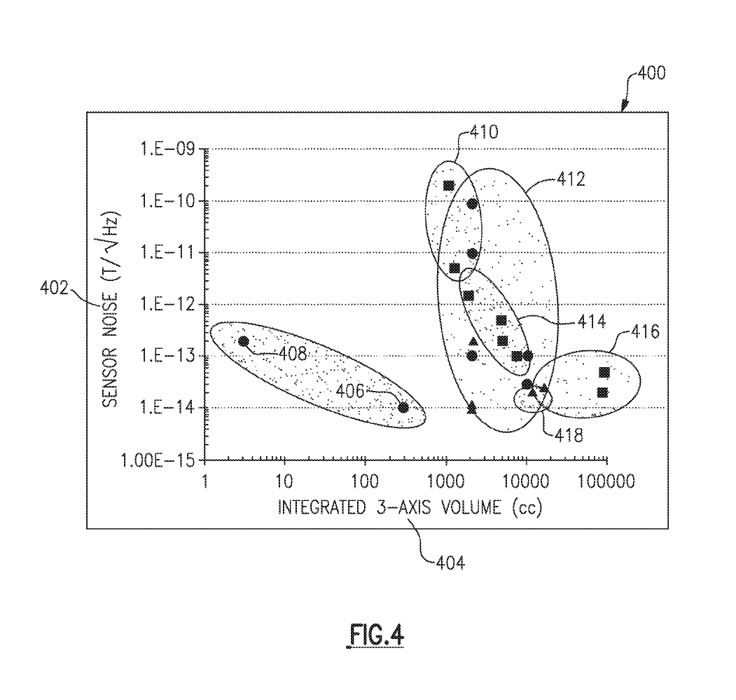

FIG. 4 provides a graph demonstrating the improved noise insensitivity of various examples when compared with known magnetic field detectors. In particular, FIG. 4 demonstrates a comparison of the noise at 1 Hz. In FIG. 4, the vertical axis 402 represents the sensor system resolution (T/√Hz) and the horizontal axis 404 represents sensor volume (cc). As discussed above, magnetic field detectors are typically limited by the total noise that contributes to the measurement of the magnetic field. The detector, and the natural and human environment (e.g., Earth's background magnetic field), all contribute to this total noise. Accordingly, total noise determines the system resolution (T/√Hz).

In contrast to conventional detectors, various embodiments provide an ultra-low noise detector which can observe weak magnetic field signals of interest. The same result is challenging to achieve with high noise detectors because the signal of interest is often indistinguishable from noise in the system. As demonstrated in FIG. 4, not only do various embodiments exhibit improved sensitivity, for equivalent levels of sensitivity, embodiments are volumetrically much smaller. Examples of the noise sensitivity of various known detectors are represented by data groups 410, 412, 414, 416, 418, and two example points of the performance capabilities of the described magnetic field detectors are represented by the points 406 and 408.

Referring now to FIG. 5, illustrated is a plan view of the sense electrodes 502a, 502b (collectively “sense electrodes 502”) and drive electrodes 504a, 504b (collectively “drive electrodes 504”) of the magnetic field detector 100 illustrated in FIGS. 1 and 2. In particular, FIG. 5 illustrates the electrical connections between the sense electrodes 502 and the corresponding electrical contacts 116, and the electrical connections between the drive electrodes 504 and the corresponding electrical contacts 116. As previously discussed, leads 118 may couple electrical contacts 116 on the substrate 122 to electrical contacts 116 on the baseplate 114. The electrical contacts 116 may couple the detector 100 to the control circuit. For the convenience of illustration, leads 118 are not shown in FIG. 5. As discussed above with reference to FIGS. 1 and 2, in various examples the sense electrodes 502 and the drive electrodes 504 are formed on the substrate 122, and in particular, within the substrate offset space beneath the proof mass 102. FIG. 5 is described with continuing reference to the magnetic field detector 100 illustrated in FIGS. 1 and 2, and the components thereof.

FIG. 5 illustrates a first sense electrode 502a (e.g., a left sense electrode), a second sense electrode 502b (e.g., a right sense electrode), a first drive electrode 504a (e.g., a left torquer), and a second drive electrode 504b (e.g., a right torquer). As further discussed with reference to FIG. 7A-7C and FIGS. 8A-8C, each of the first sense electrode 502a, second sense electrode 502b, first drive electrode 504a, second drive electrode 504b, and electrical contacts 116 may be applied as a metallization layer to the substrate 122. For instance, each sense electrode 502, each drive electrode 504, and/or each electrical contact 116 may be a layer of chrome, platinum, or gold on the substrate 122. As previously described, one or both of the sense electrodes 502 may be used to measure a change in capacitance (e.g., electrical capacitance) relative to the proof mass 102 as a result of torsional movement of the proof mass 102. One or both of the drive electrodes 504 may be used to produce a feedback torque on the proof mass 102 and reposition the proof mass 102.

In one example, the two sense electrodes 502a, 502b are used for a differential capacitance measurement, and the two drive electrodes 504a, 504b are used as torquers for force feedback during closed loop operation. Each sense electrode 502 and drive electrode 504 is interposed between a pair of respective electrical contacts 116 and extended along a length of the substrate 122. While shown in FIG. 5 as a pair of sense electrode plates and a pair of drive electrode plates, each plate having a substantially rectangular shape, in various other examples any suitable number of sense electrodes 502 and drive electrodes 504 may be used, and each of the sense electrodes 502 or drive electrodes 504 may have any suitable shape. Moreover, in certain examples the first sense electrode 502a and the first drive electrode 504a may be connected and act as a single large electrode to maximize performance when not operating in a closed loop mode of operation. In such an example, the second sense electrode 502b and the second drive electrode 504b may be coupled in a similar manner. In certain examples, the sense electrodes 502 and the drive electrodes 504 may be reversed and their relative areas chosen to optimize the relative level of performance between the drive and sense operations. In one example, the sense electrodes 502a, 502b (e.g., the outer positioned electrodes) act on the plurality of supports 106 of the detector 100, and therefore may have a greater effectiveness.

In various examples, each sense electrode 502 and each drive electrode 504 may include a respective guard ring 506. As shown in FIG. 5, the proof mass 102 may also have a guard ring 508. Each guard ring 506 substantially surrounds the respective sense electrode 502 or drive electrode 504 and separates that sense electrode 502 or drive electrode 504 from the other sense electrode 502 and drive electrode 504. In one example, each the guard ring 506 is a thin metal track that traces the perimeter of the corresponding plate or electrode. Each guard ring 506, 508 substantially eliminates direct-current (DC) current and low-frequency leakage currents from unintentionally effecting the corresponding sense electrodes 502, drive electrodes 504, or proof mass 102. DC current and low-frequency leakage current may limit the dynamic range of the magnetic field detector 100 and may create low-frequency noise by producing undesired voltages in the source impedances. FIG. 5 further shows a ground contact 510 for the proof mass 102.

Turning now to FIG. 6, shown is one example of a control circuit 600 that may be coupled to the magnetic field detector 100 illustrated in FIGS. 1 and 2 to detect the characteristics of a magnetic field received at the detector 100 and/or provide one or more control signals (e.g., for driving the drive electrodes). For instance, the control circuit 600 may be coupled to the contacts 116 illustrated in FIGS. 1 and 2. FIG. 6 is discussed with continuing reference to the magnetic field detector 100 of FIGS. 1 and 2, and the components thereof.

In certain examples, the control circuit 600 may include any processor, multiprocessor, or controller. The processor may be connected to a memory and a data storage element. The memory stores a sequence of instructions coded to be executable by the processor to perform or instruct the various components discussed herein to perform the various processes and acts described herein. For instance, the control circuit 600 may communicate with, and provide one or more control signals to the sense electrodes and the drive electrodes of the magnetic field detector via the contacts 116 and the leads 118. The memory may be a relatively high performance, volatile random access memory such as a dynamic random access memory (DRAM) or static memory (SRAM). However the memory may include any device for storing data, such as a disk drive or other nonvolatile storage device.

The instructions stored on the data storage may include executable programs or other code that can be executed by the processor. The instructions may be persistently stored as encoded signals, and the instructions may cause the processor to perform the functions and processes described herein, such as providing one or more control signals to generate a feedback torque. The data storage may include information that is recorded, on or in, the medium, and this information may be processed by the processor during execution of instructions. The data storage includes a computer readable and writeable nonvolatile data storage medium configured to store non-transitory instructions and data. In addition, the data storage includes processor memory that stores data during operation of the processor.

In the illustrated example, the control circuit 600 includes a precision square-wave generator 602 which is coupled to a first filter 604. The precision square-wave generator 602 generates a signal which is converted to a sine wave by the first filter 604. The first filter 604 may include any suitable filter designed to accept a square-wave input and provide a sinusoidal output. For instance, one example is a low-Q active bandpass filter with a notch filter to reduce the third-order harmonic. In various examples, the first filter 604 has a very low amplitude sensitivity to temperature, such as 1-3 ppm per degree Celsius. The first filter 604 is coupled to an inverting amplifier 606 which has an adjustable gain and a nominal gain of −1. Accordingly, an output of the first filter 604 and the inverting amplifier 606 form a low-noise differential sine-wave carrier generator.

As shown in FIG. 6, the carrier generator may be coupled to each of the sense electrodes (e.g., shown as readout capacitors 608a, 608b, collectively “readout capacitors 608”) to excite the readout capacitors 608 in order to up-convert (e.g., increase a frequency) an electronics signal produced by the received magnetic field. In various examples, by up-converting the received magnetic field information, the information is converted to a frequency where amplifier noise is significantly lower. Moreover, the up-conversion reduces the sensitivity of the magnetic field to current noise sources in a preamplifier 610 coupled to the readout capacitors 608. While not illustrated in FIG. 6, in many instances the control circuit 600 may include one or more passive high-pass filters interposed between the outputs of the carrier generator and the readout capacitors 608 to reduce low-frequency voltage noise coupled to the readout capacitors 608 from the carrier generator. Such an arrangement offers the benefit of reduced low-frequency torque noise.

Referring to the magnetic field detector 100 of FIG. 1, in the absence of a magnetic field, there will be no torque on the proof mass 102 (in an ideal case). In such a situation, no magnetic field information is passed from the readout capacitors 608 (sense electrodes 502 in FIG. 5) to the preamplifier 610. However, when a magnetic field is present, the readout capacitors 608 provide a measured signal to the preamplifier 610, which in turn provides an output of a carrier signal amplitude-modulated by the magnetic field (e.g., a double-sideband suppressed carrier signal).

In various examples, the control circuit 600 includes a second amplifier 612 and a second filter 614 coupled to the output of the preamplifier 610. For instance, the second amplifier 612 may include a low-noise instrumentation amplifier with an input-referred noise density that is substantially less than the output-referred noise density. The carrier signal amplitude-modulated by the magnetic field is received and amplified by the second amplifier 612 before being filtered by the second filter 614 and received at a demodulator 618. According to certain examples, the second filter 614 includes a band-pass filter which has a low quality factor to reduce the noise within the amplitude-modulated carrier signal at the third order and higher order harmonics. Accordingly, the second filter 614 provides filtering functionality to prevent higher order harmonics from affecting the noise performance of the control circuit 600 after the carrier signal has been demodulated. In certain implementations, the control circuit 600 may also include a third amplifier 616 which is coupled to an output of the second filter 614 and configured to add an additional gain to the carrier signal amplitude-modulated by the magnetic field information. While illustrated in FIG. 6 as separated from the second filter 614, in certain examples the third amplifier 616 provides additional AC gain and may be incorporated into the second filter 614.

As shown in FIG. 6, the control circuit 600 includes a demodulator 618 and comparator 620 which are coupled to form a switching (or square-wave) demodulator. In FIG. 6, the switching demodulator is coupled to an output of the third amplifier 616. The demodulator 618 drives a controller 622, which is coupled to the output of the demodulator 618. In some examples, the controller 622 may include an Integral-Derivative (ID) controller, a Proportional-Integral-Derivative (PID) controller, or any other suitable predictive controller. In one example, the controller 622 drives a torque generator 624 which produces a bias voltage at each respective torque generator electrode (e.g., drive electrodes 504a, 504b illustrated in FIG. 5). In particular, the torque generator may produce respective torque generator voltages of (BIAS+K*V_{C}) and (BIAS−K*V_{C}), where “BIAS” is a bias voltage, “K” is a scaling constant, and “V_{C}” is the output of the controller 622. For example, the torque generator 624 may produce a substantially constant bias voltage having a nominal value near one-half of the positive or negative supply voltage. While in the illustrated example, the torque generator 624 includes summation blocks 634, 638, an inverting gain 636, and an adjustable gain 632 for the purpose of illustration, in various other examples the torque generator 624 may be implemented with various other suitable components.

Accordingly, the applied torque, which is proportional to the square of the voltage, is directly proportional to the output of the controller 622. Such a biasing arrangement achieves a linearization of the closed-loop feedback torque applied to the proof mass 102 with respect to the output of the controller 622. This arrangement results in a linear control loop and permits a linear readout of the magnetic field information. In certain examples, the control circuit 600 may further include one or more passive low-pass filters (not shown) interposed between the torque generator 624 and the torque generator electrodes in order to reduce carrier-band noise applied to the torque generator electrodes.

As further illustrated in FIG. 6, the control circuit 600 may include a baseband filter 626 coupled to the output of the controller 622. For example, the baseband filter 626 may include a bandpass filter having a passband selected to extract the magnetic field information within the desired bandwidth from the output of the demodulator 618. The output of the baseband filter 626 may then be amplified by a fourth amplifier 628 and provided to an output of the control circuit 600 or one or more downstream diagnostic electronics. In at least one example, the fourth amplifier 628 is designed such that most of a variable voltage range of the amplifier 628 corresponds to a maximum expected in-band field strength of the magnetic field. Such a design provides the benefit of reduced noise. For instance, the fourth amplifier 628 may include a high-gain amplifier that has a gain of about 100. The parameters of the fourth amplifier 628 may be selected in conjunction with the parameters of the baseband filter 626 to select and amplify a desired frequency band (e.g., a frequency band associated with brain activity (0.5 Hz-100 Hz)). As shown, in certain examples the control circuit 600 may also include a fifth amplifier 630 to provide an unfiltered output for diagnostic purposes.

Though the features within FIG. 6 are illustrated as blocks within a block diagram, unless otherwise indicated, the features may be implemented as signal processing circuitry, and may be implemented with one or more specialized hardware components or one or more specialized software components. For instance, the control circuit 600 may be implemented as one of, or a combination of, analog circuitry or digital circuitry. The control circuit 600 may be composed of an array of logic blocks arranged to perform one or more of the corresponding signal processing operations described herein. In particular, the processing circuitry may implemented by an array of transistors arranged in an integrated circuit that provides a performance and power consumption similar to an ASIC (application-specific integrated circuit) or an FPGA (field-programmable gate array). In other examples, components of the control circuit 600 may be implemented as one or more microprocessors executing software instructions (e.g., predefined routines). In particular, the software instructions may include digital signal processing (DSP) instructions. Unless otherwise indicated, signal lines may be implemented as discrete analog or digital signal lines, or as a single discrete digital signal line with appropriate signal processing to process separate signals.

Turning now to FIGS. 7A-7C and FIGS. 8A-C, illustrated is an example of a process 700 for fabricating a magnetic field detector, such as the magnetic field detector 100 illustrated in FIGS. 1, 2 and FIG. 3. In particular, FIG. 7A-7C illustrates the process flow and FIGS. 8A-8C show a state of a magnetic field detector during each act of the process 700. Each act of the process 700 of FIG. 7A-7C is illustrated immediately adjacent the corresponding state of production of the magnetic field detector. Accordingly, in some examples, the magnetic field detector shown in FIGS. 8A-8C may be one implementation of the magnetic field detector 100 described with reference to at least FIGS. 1 and 2. That is, at least the magnetic dipole source, the substrate, the support(s), the proof mass, the sense electrode(s), and the drive electrode(s) described with reference to FIGS. 8A-8C may correspond to the magnetic dipole source, the support(s), the proof mass, the sense electrode(s), and the drive electrode(s) previously described with reference to at least FIGS. 1 and 2, as well as, the sense electrode(s) and the drive electrode(s) described with reference to FIG. 5.

The process 700 begins at act 702 which may include the act of providing a substrate wafer 802 (referred to generally as the “substrate 802”). In various examples, the substrate 802 is a glass wafer. The glass wafer may be doped such that it conducts electricity at elevated temperatures (e.g., about 350 degrees Celsius). The glass wafer may be composed of borosilicate, for example. In act 704, the process 700 includes defining a well 804 (e.g., a substrate offset space) in the substrate 802. In certain examples, the substrate offset space is formed by etching the substrate 802; however, other processing techniques may be used, such as milling, grinding, or one or more deposition processes. For instance, the etching process may be implemented using the MESA™ etch system offered by APPLIED MATERIALS™ of Santa Clara, Calif. Areas of the substrate 802 which are not etched during act 704 may be later coupled to a flexure layer 814 or a handle layer 816 of a structure wafer 812, as discussed below.

In act 706, the process 700 may include depositing a conducting material, such as metal, on the substrate 802 to form one or more sense electrodes 806, one or more drive electrodes 808, and/or one or more guard rings and electrical contacts (not shown in FIG. 8A). In the shown example, the conducting material is primarily deposited on the substrate 802 and within the substrate offset space 804. For instance, each sense electrode 806 and each drive electrode 808 may be formed on a surface of the substrate 802 within the substrate offset space 804. As discussed with reference to FIGS. 1 and 2, each sense electrode 806 may be configured to measure a change in capacitance within the substrate offset space 804 (e.g., between the sense electrode and a proof mass), and each drive electrode 808 may be configured to act as a closed loop torquer on the proof mass. Each guard ring is formed on the substrate 802 to substantially surround a corresponding one of the sense electrodes 806 or drive electrodes 808, and isolates that respective sense or drive electrode plates 806, 808 from the effects of direct-current (DC) current and low-frequency leakage currents.

In act 708, the process 700 may include conditioning the surface(s) of one or more sense electrodes 806 and/or drive electrodes 808 to increase the surface texture thereof. In one example, act 708 may include applying one or more small metal bumps 810 to the surface of the sense electrodes 806 and/or drive electrodes 808. The increase in surface texture decreases the holding force between the substrate 802 and the structure wafer 812 by reducing the contact area between the substrate 802 and the structure wafer 812.

In act 710, the process 700 may include providing a structure wafer 812, such as a Silicon-on-Insulator (SOI) wafer. While a SOI wafer is used as one example for the purpose of explanation, in various other examples, other suitable structure wafer materials may be used, such as quartz, polysilicon, etc. In the shown example of FIGS. 8A-8C, the structure wafer 812 includes a flexure layer 814 and a handle layer 816 separated by a buried oxide layer 818. In one example, the flexure layer 814 is about 400 μm thick (e.g., ±2 μm thickness), the handle layer 816 is about 300 μm thick (e.g., ±2 μm thickness), and the buried oxide 818 is about 2 μm thick (e.g., ±1 μm thickness).

Referring to FIG. 7B and FIG. 8B, in act 712 the process 700 may include defining a proof mass 820, a plurality of supports 822, and/or one or more anchors 824 in the structure wafer 812. In the shown example of FIG. 8B, each support 822 is interposed between the proof mass 820 and a respective anchor 824. In certain examples, the proof mass 820, the plurality of supports 822, and/or the one or more anchors 824 are formed by etching the flexure layer 814 of the structure wafer 812; however, other processing techniques may be used, such as milling, grinding, or one or more deposition processes. In certain examples, a Deep Reactive Ion Etch (DRIE) process may be used with a dry etch tool and Inductively Coupled Plasma (ICP) to define each of the proof mass 820, supports 822, and the anchors 824. In one example, the ICP etch may also define one or more holes in the flexure layer 814. Each hole may be used to electrically connect the flexure layer 814 and the handle layer 816, as described during later processing acts of FIG. 7A-7C. In FIG. 7B, the flexure layer 814 is shown as having a hole 832a within the proof mass 820 and a hole 832b, 832c within each anchor 824.

In act 714, the process 700 may include selectively removing a first portion of the oxide layer 818 from the structure wafer 812. In particular, the first portion may include those areas of the oxide layer 818 that were exposed during the etching process of act 712. That is, in one example, act 714 may include removing the exposed oxide from the holes 832a, 832b, 832c in the flexure layer 814. For instance, an oxide ICP etch may be used to remove the exposed oxide. Following act 714, in act 716 the process 700 may include defining one or more counterbalances in the handle layer 816 of the structure wafer 812. For instance, act 716 may include etching the handle layer 816 to define a counterbalance 826 for the proof mass 820. In act 716, the process 700 may further include defining one or more anchor grounds 834. Each anchor ground 834 couples a respective anchor 824 to the substrate 802, as further discussed below with reference to act 722.

In act 718, the process 700 may include selectively metallizing each recess (e.g., hole) formed in the flexure layer 814 of the structure wafer 812 to plate the one or more formed recesses. The deposited metal 828 forms an electrical connection between the flexure layer 814 and the handle layer 816. Following act 718, in act 720 the process 700 includes the act of etching a second portion of the oxide layer 818. As shown in FIG. 8B, the second portion of the oxide layer 818 may include those sections of the oxide layer 818 that are attached to the supports 822. Accordingly, act 720 may include releasing the supports 822 from the oxide layer 818 to suspend the proof mass 820. In at least one example, the supports 822 are released by removing the second portion of the oxide layer 818 using a Hydrofluoric acid (HF) etching process.

Once each of the supports 822 has been released, the process 700 may include coupling the structure wafer 812 to the substrate 802, as shown in FIG. 8C. In one example, the handle wafer 816 may be anodically bonded to the substrate 802 (act 722). Once the structure wafer 812 has been coupled to the substrate 802, the proof mass 820 may be suspended above and partially within the substrate offset space 804 by the plurality of supports 822. The anchor grounds 834 may couple the flexure layer 814 to the substrate 802 at each end of the flexure layer 814 (e.g., at each anchor 824), where the substrate offset space 804 is substantially in the center of the substrate 802. In an example where multiple magnetic field detectors are fabricated from the same of substrate 802 material and structure wafer 812 (e.g., SOI wafer), the process 700 may then include dicing each sheet to separate each of the separate magnetic field detectors. The process 700 ends in act 724, in which a magnetic dipole source 830 is coupled to the structure wafer 812, and in particular, coupled to the proof mass 820. As shown, the magnetic dipole source 830 is positioned at about the center of the flexure layer 814 such that each of the supports 822 suspends the magnetic dipole source 830 above the substrate offset space. Processes and acts for operating the magnetic field detector once it has been fabricated are discussed above with reference to the magnetic field detector 100 shown in at least FIGS. 1 and 2.

As discussed above, in various examples the assembled magnetic field detector may be packed with a housing, a baseplate, and one or more electrical connections, such as the housing 110 and the baseplate 114 illustrated in FIGS. 1 and 2, and the electrical connections illustrated in FIG. 5. In various examples, the magnetic dipole source 830 may be coupled to the flexure layer 814 early in the packaging process (e.g., before the sense electrodes 806 and/or drive electrodes 808 are electrically bonded to the substrate 802).

As such, in addition to providing improved magnetic field detectors that exploit the magnetic component of electromagnetic signals, various other aspects and examples discussed herein provide improved fabrication processes for efficiently and cost-effectively producing a compact magnetic field detector. Particular examples of the magnetic field detector may include a magnetic field detector capable of detecting bio-physical signals generated by the body of a patient or user, such as the magnetic field of his or her brain, heart, nerves or muscles. When compared to available electromagnetic sensors examples of the magnetic field detector herein achieve a low noise (e.g., less than 1 pT/rtHz at 10 Hz) at a compact size (e.g., less than 1 cm³) and a low production cost.

Having described above several aspects of at least one embodiment, it is to be appreciated various alterations, modifications, and improvements will readily occur to those skilled in the art. Such alterations, modifications, and improvements are intended to be part of this disclosure and are intended to be within the scope of the disclosure. Accordingly, the foregoing description and drawings are by way of example only, and the scope of the disclosure should be determined from proper construction of the appended claims, and their equivalents.

What is claimed is:

1. A magnetic field detector comprising:
a proof mass;
a magnetic dipole source coupled to the proof mass;
a substrate having a substrate offset space defined therein, wherein the proof mass is suspended above the substrate offset space;
a first sense electrode disposed on the substrate within the substrate offset space and positioned proximate the proof mass, the first sense electrode being configured to measure a change in capacitance relative to the proof mass from torsional movement of the proof mass in response to a received magnetic field at the magnetic dipole source; and
a control circuit coupled to the first sense electrode and configured to determine a characteristic of the magnetic field based on the measured change in capacitance.

2. The magnetic field detector of claim 1, further comprising a counterbalance coupled to the proof mass, wherein the magnetic dipole source is coupled to a first surface of the proof mass and the counterbalance is coupled to a second surface of the proof mass distal the magnetic dipole source.

3. The magnetic field detector of claim 1, further comprising a second sense electrode coupled to the control circuit, wherein the second sense electrode is disposed on the substrate, and wherein the first sense electrode and the second sense electrode are configured to provide a differential capacitance measurement based on the change in capacitance from the torsional movement of the proof mass.

4. The magnetic field detector of claim 3, further comprising at least one drive electrode coupled to the control circuit and positioned proximate the proof mass, wherein the at least one drive electrode is configured to produce a feedback torque on the proof mass.

5. The magnetic field detector of claim 4, wherein the at least one drive electrode is positioned on the substrate and within the substrate offset space.

6. The magnetic field detector of claim 5, further comprising a plurality of guard rings, each guard ring positioned to substantially surround a corresponding one of the first sense electrode or the at least one drive electrode.

7. The magnetic field detector of claim 1, wherein the magnetic dipole source is formed from at least one of a static permanent magnet and an electromagnet.

8. The magnetic field detector of claim 1, further comprising at least one support coupled to the proof mass and configured to suspend the proof mass above the substrate offset space.

9. The magnetic field detector of claim 8, further comprising a structure wafer, wherein at least the proof mass and the at least one support are defined in the structure wafer.

10. The magnetic field detector of claim 9, wherein the structure wafer is a Silicon-on-Insulator (SOI) wafer having a flexure layer, a handle layer, and an oxide layer, the oxide layer being interposed between the flexure layer and the handle layer, and wherein the proof mass and the at least one support are defined in the flexure layer.

11. The magnetic field detector of claim 1, further comprising a levitation suspension system configured to levitate the proof mass relative to the substrate.

12. The magnetic field detector of claim 11, wherein the levitation suspension system includes at least one levitation forcer positioned proximate the proof mass and configured to apply a force to maintain the proof mass at a null point, and wherein the at least one levitation forcer is an electrostatic forcer or a magnetic forcer.

13. The magnetic field detector of claim 1, wherein the magnetic dipole source is configured to generate a dynamic magnetic dipole, the control circuit being configured to provide an induced voltage to vary the dynamic magnetic dipole.

14. The magnetic field detector of claim 1, further comprising an auxiliary sensor coupled to the control circuit and configured to measure an external parameter, the external parameter including at least one of noise, a vibration, and an ambient temperature, and wherein the control circuit is configured to adjust the characteristic of the magnetic field to compensate for an effect of the measured external parameter on the characteristic of the magnetic field.

15. The magnetic field detector of claim 1, wherein the control circuit includes a preamplifier, a demodulator, and a baseband filter, and wherein the preamplifier is configured to provide a carrier signal amplitude-modulated by the magnetic field and the demodulator is configured to receive the amplitude-modulated carrier signal, and wherein the baseband filter is configured to extract the characteristic of the magnetic field from an output of the demodulator.

16. The magnetic field detector of claim 1, wherein the control circuit is further configured to apply a bias voltage and create a negative spring force on the proof mass.

17. A magnetic field transduction method comprising:
generating a magnetic dipole on a proof mass, the proof mass being suspended above a substrate offset space in a substrate relative to a first sense electrode disposed on the substrate;
measuring a change in capacitance between the first sense electrode and the proof mass from torsional movement of the proof mass in response to receiving a magnetic field at the proof mass; and
determining a characteristic of the magnetic field based on the measured change in capacitance.

18. The method of claim 17, further comprising providing a differential capacitance measurement from the first sense electrode and a second sense electrode based on the change in capacitance from the torsional movement of the proof mass.

19. The method of claim 17, further comprising suspending the proof mass relative to the first sense electrode with at least one of one or more supports, one or more rotational bearings, an electrostatic suspension, or a magnetic suspension.

20. The method of claim 19, further comprising providing a feedback torque on the proof mass with one or more drive electrodes positioned proximate the proof mass.

21. The method of claim 17, wherein generating the magnetic dipole includes forming the magnetic dipole on the proof mass with a permanent magnet or an electromagnet.

22. A method of fabricating a magnetic field detector comprising:
defining at least one substrate offset space in a substrate wafer;
forming a first sense electrode on the substrate wafer and within the substrate offset space;
defining a proof mass and at least one support in a structure wafer and suspending the proof mass by the at least one support to allow torsional movement of the proof mass;
providing a magnetic dipole source on the proof mass; and
coupling the substrate wafer and the structure wafer to position the proof mass proximate the substrate offset space of the substrate wafer and within capacitive communication with at least the first sense electrode.

23. The method of claim 22, further comprising providing the structure wafer, wherein the structure wafer includes a flexure layer, a handle layer, and an oxide layer, the oxide layer being interposed between the flexure layer and the handle layer, and wherein defining the proof mass and the at least one support in the structure wafer includes etching the flexure layer to form the proof mass and the at least one support.

24. The method of claim 23, further comprising applying a metallic layer to one or more holes defined in the flexure layer to electrically couple the flexure layer and the handle layer of the structure wafer.

25. The method of claim 22, further comprising forming a second sense electrode, a first drive electrode, and a second drive electrode on the substrate wafer and within the substrate offset space.
